(19) United States
(12) Patent Application Publication
(10) Pub. No.: US 2020/0390995 A1
(43) Pub. Date: Dec. 17, 2020

(54) METHOD AND DEVICE FOR IMPLANTED PORT ALLEVIATION

(71) Applicant: Mathew Valdez, Lancaster, CA (US)

(72) Inventor: Mathew Valdez, Lancaster, CA (US)

(21) Appl. No.: 16/442,948

(22) Filed: Jun. 17, 2019

(51) Int. Cl. A61M 19/00; A61M 35/00; A61M 39/02

(52) U.S. Cl. CPC A61M 19/00; A61M 35/00; A61M 2209/088; A61M 2205/0216; A61M 39/0208

(57) ABSTRACT

A device and method for implanted port alleviation. The device including a dispensing cap having a compressible reservoir and a cover member, wherein the compressible reservoir is configured to hold a predetermined quantity of a topical anesthetic and the cover member is configured to cover an implanted port on the patient. An adhesive element attached to a top portion of the compressible reservoir configured to adhere to the skin of a patient. Advantageously, the compressible reservoir is positioned on top of the cover member such that when the compressible reservoir is compressed, the topical anesthetic is transferred to the cover member on the patient's skin above the implanted port numbing the patient's skin.

### CROSS-REFERENCE TO RELATED APPLICATIONS

N/A

### BACKGROUND OF THE INVENTION

### 1. Field of the Invention

The present invention relates generally to medical devices but more particularly to a method and device for implanted port alleviation.

### 2. Description of Related Art

Patients undergoing intensive medical treatment may be required to have repeated injections of drugs or multiple drawing of blood samples. To ease patient discomfort associated with repeated needle stick, the patient may elect to have a port surgically implanted beneath the skin. Various types of implantable ports are available and go by various names, such as Port-A-Cath®, BardPort®, PassPort®, Medi-port®, and Infusaport®. Additionally, ports are a form of a “central venous catheters” or “central venous access device” and are frequently referred to as such in the medical field, as well as “implantable venous access ports.”

Ports are usually implanted in the chest, but may be located elsewhere. The ports may stay in place for many weeks, months, or even years. Generally, the ports are constructed of a small circular drum usually made plastic or metal having a thin tube going from the drum into a large vein. The drum has a silicone septum across the top enabling needles to enter the septum to use the port.

Frequently, patients experience discomfort and pain when their port is accessed. Prescription creams and topical anesthetic are often provided to patients to help alleviate pain. These are generally applied about 30 minutes prior to their ports being accessed. However, there are many problems with these current methods. Often times, patients don't place enough topical anesthetic on the port as the topical either rubs off on the patient's clothes, or is absorbed in the saran wrap which is sometimes used to cover the port after applying the topical. Thus, the patients still experience pain and discomfort during access. Consequently, there is a need for an improved method and/or device to reduce pain when a port is accessed.

### BRIEF SUMMARY OF THE INVENTION

It is an object of the present invention to provide a device and method for reducing a patient's pain and discomfort when their implanted port is accessed. In order to do so, in one aspect of the it on a device for implanted port alleviation is provided, comprising a dispensing cap having a compressible reservoir and a cover member, wherein the compressible reservoir is configured to hold a predetermined quantity of a topical anesthetic and the cover member is configured to cover an implanted port on the patient; an adhesive element attached to a top portion of the compressible reservoir, wherein the adhesive element is configured to adhere to the skin of a patient; and, the compressible reservoir positioned on top of the cover member such that when the compressible reservoir is compressed, the predetermined quantity of topical anesthetic is transferred to the cover member on the patient's skin above the implanted port numbing the patient's skin.

In another aspect of the invention, a method for implanted port alleviation is provided comprising steps: (a) providing a device including a dispensing cap having a compressible reservoir, a cover member, and an adhesive element, wherein the compressible reservoir is configured to hold a predetermined quantity of a topical anesthetic; (b) placing the cover member over an implanted port on a patient's skin; (c) adhering the adhesive element to the patients skin, and, (d) compressing the compressible reservoir such that the predetermined quantity of topical anesthetic is transferred to the cover member numbing the patient's skin above the implanted port.

In one embodiment, the cover member is circular. In one embodiment, the cover member is sized to cover the implanted port when the device is secured to the patient. In one embodiment, the topical anesthetic is lidocaine. In one embodiment, the adhesive element contains an adhesive substance to the underside of the adhesive element. In another embodiment, the adhesive element is constructed from an elastic material.

### BRIEF DESCRIPTION OF THE SEVERAL VIEWS OF THE DRAWINGS

Other features and advantages of the present invention will become apparent when the following detailed description is read in conjunction with the accompanying drawings, in which:

FIG. 1 is a perspective view showing an implanted port in a patient;

FIG. 2 is a perspective view of a device for implanted port alleviation with the adhesive element in the open position according to an embodiment of the present invention;

FIG. 3 is a perspective view of the device for implanted port alleviation prior to use according to an embodiment of the present invention; and,

FIG. 4 is a perspective view of the device and method for implanted port alleviation is use according to an embodiment of the present invention.

### DETAILED DESCRIPTION OF THE PREFERRED EMBODIMENT

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein to specifically provide a device and method for implanted port alleviation.

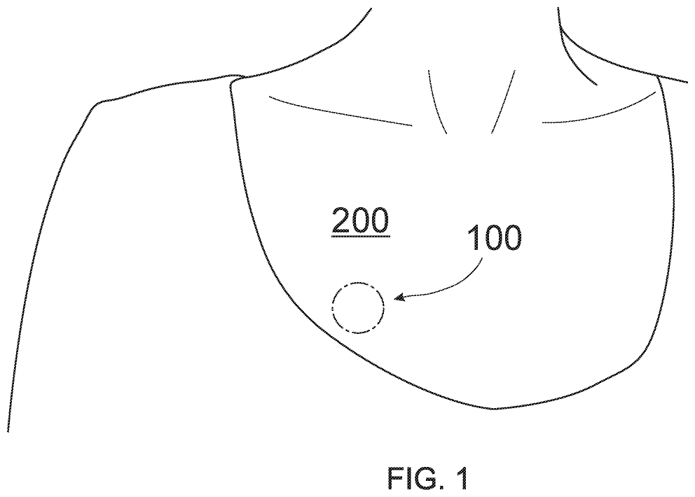

FIG. 1 shows an implanted port 100 in a patient 200 according to an embodiment of the present invention. Referring now to FIG. 1, the implanted port 100 is shown implanted under the skin on the chest of the patient 200. It should be understood that although the implanted port 100 is positioned in the chest, the port may be located in any position on the patient 200.

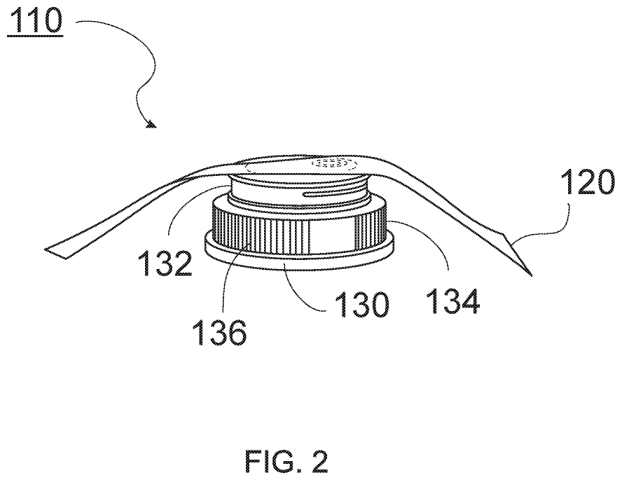

FIG. 2 is a perspective view of a device 110 for implanted port alleviation with the adhesive element 120 in the open position according to an embodiment of the present invention. Referring now to FIG. 2, the device 110 comprises adhesive element 120 and dispensing cap 130. In one embodiment, a portion of the adhesive element is attached to the top portion of the dispensing cap 130. In a preferred embodiment, the dispensing cap 130 includes a compressible reservoir 132 and cover member 134. The cover member 134 and the compressible reservoir 132 are both hollow, wherein the compressible reservoir 132 is configured to hold a predetermined quantity of a topical anesthetic. In one embodiment, the dispensing cap 130 and particularly, the cover member 134 is circular and configured to match the shape of the implanted port 100 (FIG. 1). However, the dispensing cap 130 and/or the cover member 134 may be any shape known the art, without departing from the spirit and scope of the invention. Similarly, the cover member 134 is sized to be slightly larger than the implanted port 100. In some embodiments, grip traction 136 may be provided on any portion of the device 110 to increase the ease of handling and operating the device 110.

In one embodiment, the compressible reservoir 132 is prefilled with the predetermined quantity of a topical anesthetic, and the device 110 is configured to be a one-time use product. However, in some embodiments the compressible reservoir 132 may be filled by the patient or a qualified user such that the device 110 is reusable. During use, the compressible reservoir 132 is compressed by the patient to release the predetermined quantity of the topical anesthetic into the cover member 134 of the dispensing cap 130 such that the predetermined quantity of the topical anesthetic is situated on the patient's skin above the implanted port 100, this will be discussed in further details below.

The means and structure of how the compressible reservoir 132 functions may be any method and/or structure known in the art. The critical aspect is that the predetermined quantity of the topical anesthetic is transferred from the compressible reservoir 132 into the cover member 134 when the compressible reservoir 132 is compressed by the patient. Thus, the structure of how compressible reservoir 132 functions may vary. Further, the structure of how the compressible reservoir 132 functions may depend on if the device 110 is reusable or is a one-time use product. Some examples may include, but are not limited to, a pierceable film that is pierced when compressed, a flexible material having perforations that open when compressed, and a resealable membrane situated between the compressible reservoir and the cover member configured to be unseated when compressed.

In the preferred embodiment, the topical anesthetic is lidocaine. However, other anesthetics may be used including but not limited to diphenhydramine, chloroprocaine, benzocaine, ropivacaine, mepivacaine, procaine, prilocaine, or similar. As well known in the art, patient allergies and sensitivities may lead to one anesthetic over another.

In one embodiment, the adhesive element 120 contains an adhesive substance to the underside of the adhesive element. The adhesive may be any adhesive substance known in the art, such as acrylate. In some embodiments, the adhesive element 120 is constructed from an elastic material including but not limited to woven fabric, plastic, and latex. In one embodiment, the adhesive element 120 is generally rectangular in length, wherein the length is adequate and provides enough adhesive to secure the device 110 to the patient during use. The use of the device 110 will be described in further detail below.

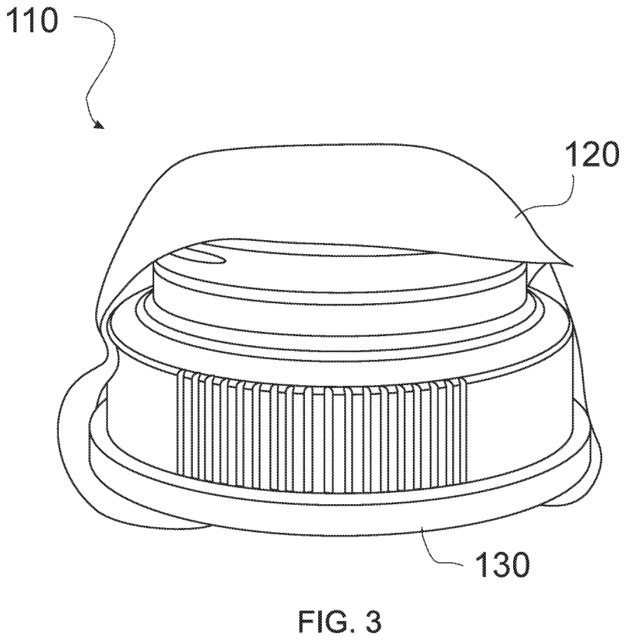

FIG. 3 is a perspective view of the device 110 for implanted port alleviation prior to use according to an embodiment of the present invention. Referring now to FIG. 3, the device 110 is shown with the adhesive element 120 wrapped around the dispensing cap 130. This folded and compact arrangement is how the device 110 would be presented and provided to the patient in the preferred embodiment. In some embodiments, a removable cover (not shown) would be provided on the bottom of the dispensing cap 130 to prevent containments from entering the dispensing cap. In a similar fashion, the device 110 may be provided is sealed package to ensure the device 110 is sterile.

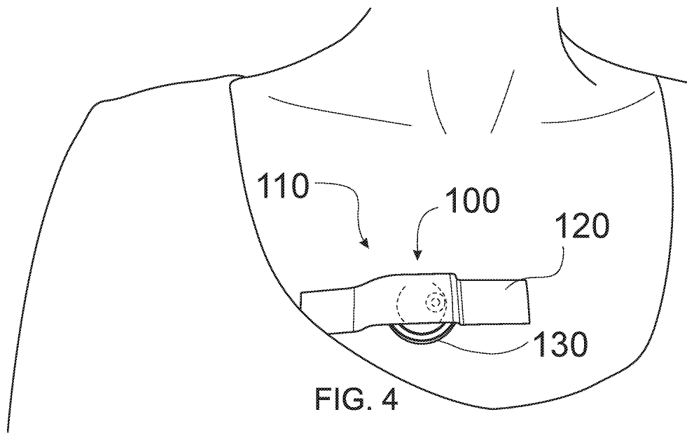

FIG. 4 is a perspective view of the device 110 and method for implanted port alleviation is use according to an embodiment of the present invention. Referring now to FIG. 4, when implanted port alleviation is desired, the patient removes the device 110 from the sealed packaging (if applicable), then unfolds the adhesive element 120 from an orientation as discussed and shown in FIG. 3. Next, the patient aligns the dispensing cap 130 and more particularly, the cover member 134 over the implanted port 100, then adheres the adhesive element 120 to the skin surrounding the implanted port 100 such that the device 110 is secured to the patient. Now the device 110 is ready for use. Next, approximately 30 minutes prior to the implanted port being accessed, the patient compresses the compressible reservoir 132 releasing the predetermined quantity of topical anesthetic into the cover member 134 such that the topical anesthetic is applied to the patient's skin above the implanted port 100 numbing the area to alleviate pain and discomfort when the implanted port 100 is accessed by medical staff.

In some embodiments, the device 110 further comprises at least one member of an identification set consisting of: an alphanumeric identification, symbolic shape, a company brand, a numeric identification number, a QR code, a barcode, and an RFD tag. The identification set may be used to identify, track, and/or provide information to the patient.

Although the invention has been described in considerable detail in language specific to structural features, it is to be understood that the invention defined in the appended claims is not necessarily limited to the specific features described. Rather, the specific features are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention.

It should further be noted that throughout the entire disclosure, the labels such as left, right, front, back, top, bottom, forward, reverse, clockwise, counter clockwise, up, down, or other similar terms such as upper, lower, aft, fore, vertical, horizontal, oblique, proximal, distal, parallel, perpendicular, transverse, longitudinal, etc. have been used for convenience purposes only and are not intended to imply any particular fixed direction or orientation. Instead, they are used to reflect relative locations and/or directions/orientations between various portions of an object.

In addition, reference to “first,” “second,” “third,” and etc. members throughout the disclosure (and in particular, claims) are not used to show a serial or numerical limitation but instead are used to distinguish or identify the various members of the group.

What is claimed is:

1. A device for implanted port alleviation comprising:
a dispensing cap having a compressible reservoir and a cover member, wherein the compressible reservoir is configured to hold a predetermined quantity of a topical anesthetic and the cover member is configured to cover an implanted port on the patient;
an adhesive element attached to a top portion of the compressible reservoir, wherein the adhesive element is configured to adhere to the skin of a patient; and, the compressible reservoir positioned on top of the cover member such that when the compressible reservoir is compressed, the predetermined quantity of topical anesthetic is transferred to the cover member on the patient's skin above the implanted port numbing the patient's skin.

2. The device of claim wherein the cover member is circular.

3. The device of claim 1, wherein the cover member is sized to cover the implanted port when the device is secured to the patient.

4. The device of claim 1, wherein the topical anesthetic is lidocaine.

5. The device of claim 1, wherein the adhesive element contains an adhesive substance to the underside of the adhesive element.

6. The device of claim 1, wherein the adhesive element is constructed from an elastic material.

7. A method for implanted port alleviation comprising steps:
(a) providing a device including a dispensing cap having a compressible reservoir, a cover member, and an adhesive element, wherein the compressible reservoir is configured to hold a predetermined quantity of a topical anesthetic;
(b) placing the cover member over an implanted port on a patient's skin;
(c) adhering the adhesive element to the patient's skin; and,
(d) compressing the compressible reservoir such that the predetermined quantity of topical anesthetic is transferred to the cover member numbing, the patient's skin above the implanted port.

8. The method of claim 7, wherein the cover member and the implanted port is circular.

9. The method of claim 7, wherein the cover member is sized to cover the entirety of implanted port.

10. The method of claim 7, wherein the topical anesthetic is lidocaine.

11. The method of claim 7, wherein the adhesive element contains an adhesive substance to the underside of the adhesive element.

12. The method of claim 7, wherein the adhesive element is constructed from an elastic material.
